(19) United States
(12) Patent Application Publication
(10) Pub. No.: US 2026/0134989 A1
(43) Pub. Date: May 14, 2026

(54) RECEPTION DEVICE, INFORMATION PROCESSING METHOD, AND NON-TRANSITORY COMPUTER-READABLE MEDIUM

(71) Applicant: NIHON KOHDEN CORPORATION, Tokyo (JP)

(72) Inventors: Takayuki SUGIYAMA, Saitama (JP); Yuki Ayama, Saitama (JP); Hajime Shigematsu, Saitama (JP); Katsuaki Kobayashi, Saitama (JP); Mitsuyuki Amata, Saitama (JP); Takahiro Miyamura, Saitama (JP)

(73) Assignee: NIHON KOHDEN CORPORATION, Tokyo (JP)

(21) Appl. No.: 19/377,860

(22) Filed: Nov. 3, 2025

(30) Foreign Application Priority Data
Nov. 13, 2024 (JP) 2024-198050

(51) Int. Cl. G16H 40/67

(52) U.S. Cl. CPC G16H 40/67

(57) ABSTRACT

A reception device communicate with at least one transmitter. The reception device receives, from the transmitter, first specifying information, first identification information, and physiological information on a subject. The reception device stores second specifying information, and second identification information. The reception device receives the physiological information from the at least one transmitter when a communication path specified by the first specifying information matches a communication path specified by the second specifying information. When the transmitter specified by the first identification information matches the transmitter specified by the second identification information, the reception device performs a bed use process for starting monitoring of the physiological information on the subject based on information that determines that the physiological information has been transmitted from the transmitter and received by the reception device for a predetermined time or more.

### CROSS-REFERENCE TO RELATED APPLICATIONS

This application is based on and claims priority under 35 USC 119 from Japanese Patent Application No. 2024-198050 filed on Nov. 13, 2024, the contents of which are incorporated herein by reference.

### TECHNICAL FIELD

The presently disclosed subject matter relates to a reception device, an information processing method, and a non-transitory computer readable medium.

### BACKGROUND ART

JP2023-013506A discloses a patient monitor that performs channel registration by assigning a radio frequency channel based on an electric field intensity, displays a bed use screen on a display unit when the channel registration is performed, and associates the radio frequency channel for which the channel registration is performed with patient information input using the bed use screen.

In a medical facility, physiological information on a subject is monitored. In order to start the monitoring, it is necessary to perform a bed use process of the subject. The bed use process is performed, for example, by displaying the bed use screen on a central monitor and inputting the name or the like of the subject while viewing the bed use screen. However, in the related art, since the bed use process is manually performed by a medical worker, a burden on the medical worker related to the bed use process is not small, and there is room for improvement in this respect.

### SUMMARY

An object of the presently disclosed subject matter is to provide a reception device, an information processing method, a computer program, and a non-transitory computer-readable medium capable of reducing a burden on a medical worker related to a bed use process.

According to an aspect for achieving the above object, there is provided a reception device configured to communicate with at least one transmitter,
wherein the reception device is configured to receive, from the at least one transmitter, at least first specifying information for specifying a communication path used when the transmitter transmits information to the reception device using radio waves, first identification information for specifying the at least one transmitter, and physiological information on a subject associated with the at least one transmitter,
wherein the reception device is configured to store second specifying information for specifying a communication path that is used by the reception device, and second identification information for specifying the at least one transmitter that is communicated with the reception device,
wherein the reception device is configured to receive the physiological information from the at least one transmitter when the communication path specified by the first specifying information matches the communication path specified by the second specifying information, and
wherein, when the at least one transmitter specified by the first identification information matches the at least one transmitter specified by the second identification information, the reception device is configured to perform a bed use process for starting monitoring of the physiological information on the subject based on information that determines that the physiological information has been transmitted from the at least one transmitter and received by the reception device for a predetermined time or more.

According to an aspect for achieving the above object, there is provided an information processing method performed by a reception device configured to communicate with at least one transmitter, the method including:
receiving physiological information from the at least one transmitter when a communication path specified by first specifying information matches a communication path specified by second specifying information; and
performing a bed use process for starting monitoring of the physiological information on a subject based on information that determines that the physiological information has been transmitted from the at least one transmitter and received by the reception device for a predetermined time or more, when the at least one transmitter specified by first identification information matches the at least one transmitter specified by second identification information,
wherein the reception device is configured to receive, from the at least one transmitter, at least the first specifying information for specifying the communication path used when the at least one transmitter transmits information to the reception device using radio waves, the first identification information for specifying the at least one transmitter, and the physiological information on the subject associated with the at least one transmitter, and
wherein the reception device is configured to store the second specifying information for specifying the communication path that is used by the reception device, and the second identification information for specifying the at least one transmitter that is communicated with the reception device.

According to an aspect for achieving the above object, there is provided a non-transitory computer readable medium storing a computer program including at least one instruction to be executed by one or more processors of a reception device configured to communicate with at least one transmitter, when at least one instruction is executed by the one or more processors, the computer program causes the reception device to:
receive physiological information from the at least one transmitter when a communication path specified by first specifying information matches a communication path specified by second specifying information; and
perform a bed use process for starting monitoring of the physiological information on a subject based on information that determines that the physiological information has been transmitted from the at least one transmitter and received by the reception device for a predetermined time or more, when the at least one transmitter specified by first identification information matches the at least one transmitter specified by second identification information,
wherein the reception device is configured to receive, from the at least one transmitter, at least the first specifying information for specifying the communication path used when the at least one transmitter transmits information to the reception device using radio waves, the first identification information for specifying the at least one transmitter, and the physiological information on the subject associated with the at least one transmitter, and
wherein the reception device is configured to store the second specifying information for specifying the communication path that is used by the reception device, and the second identification information for specifying the at least one transmitter that is communicated with the reception device.

According to the reception device having the above configuration, the physiological information is received from the transmitter when the communication path specified by the first specifying information matches the communication path specified by the second specifying information. Then, when the transmitter specified by the first identification information matches the transmitter specified by the second identification information, the reception device performs the bed use process for starting the monitoring of the physiological information on the subject based on the information determined that the physiological information has been transmitted from the transmitter and received by the reception device for the predetermined time or more. Therefore, for example, even if the medical worker does not perform a predetermined input for performing the bed use process on the reception device, the reception device performs the bed use process when it is determined that the physiological information has been transmitted from the transmitter and received by the reception device for the predetermined time or more when the transmitter specified by the first identification information matches the transmitter specified by the second identification information. As described above, according to the reception device having the above configuration, it is possible to reduce the burden on the medical worker related to the bed use process. In addition, the information processing method, the computer program and the non-transitory computer readable medium according to the above configurations can also achieve the same or similar effects.

According to the presently disclosed subject matter, it is possible to provide a reception device, an information processing method, a computer program, and a non-transitory computer-readable medium capable of reducing a burden on a medical worker related to a bed use process.

### BRIEF DESCRIPTION OF DRAWINGS

FIG. 1 is a system configuration diagram of an information processing system according to a first embodiment.

FIG. 2 is a flowchart illustrating an information processing method performed in a first example of the first embodiment.

FIG. 3 is a flowchart illustrating an information processing method performed in a second example of the first embodiment.

FIG. 4 is a flowchart illustrating an information processing method performed in a third example of the first embodiment.

FIG. 5 is a system configuration diagram of an information processing system according to a second embodiment.

FIG. 6 is a flowchart illustrating an information processing method performed in the second embodiment.

### DESCRIPTION OF EMBODIMENTS

Hereinafter, examples of embodiments of the presently disclosed subject matter will be described with reference to the drawings.

### First Embodiment

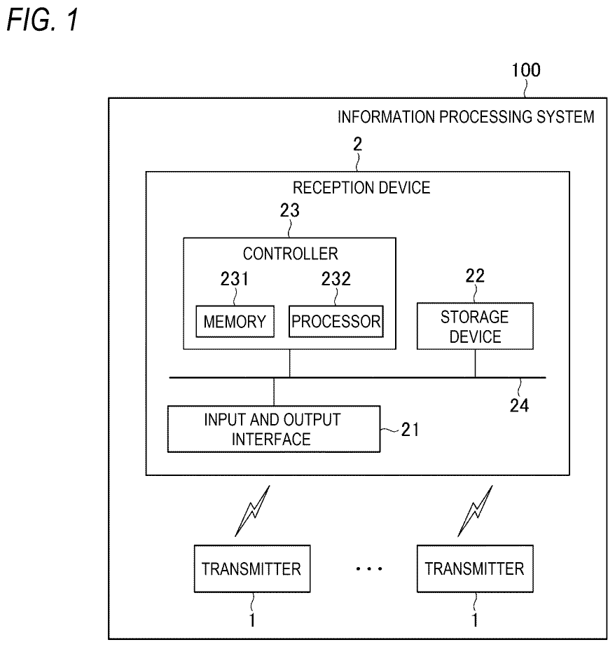

As illustrated in FIG. 1, the information processing system 100 can include a plurality of transmitters 1 and a plurality of reception devices 2. However, the information processing system 100 may include one transmitter 1. The information processing system 100 is used in a medical facility such as a hospital.

The transmitter 1 is, for example, a portable medical telemeter used by a medical worker or the like in the medical facility such as a hospital. In the present embodiment, each transmitter 1 is assigned to a specific subject. Therefore, in the medical facility such as a hospital, for example, the subject can be specified by specifying the transmitter 1. The transmitter 1 can measure and/or display physiological information on the subject. The physiological information used in the present embodiment is preferably physiological information measured continuously in time, such as electrocardiogram waveform data, percutaneous arterial oxygen saturation (SpO₂), a heart rate, and a respiratory rate. The transmitter 1 can wirelessly communicate with the reception device 2 by registering second specifying information to be described later in the reception device 2. Thus, the transmitter 1 can transmit various types of information to the reception device 2. Examples of the various types of information include physiological information measured by the transmitter 1, first specifying information for specifying a communication path used when the transmitter 1 transmits information to the reception device 2 using radio waves, and first identification information for specifying the transmitter 1.

The first specifying information is, for example, information indicating a communication path such as a channel or a frequency used when the transmitter 1 transmits information to the reception device 2 using radio waves. The channel is a path for transmitting various types of information such as physiological information, and is represented by a number such as “1001”. Therefore, the first specifying information is, for example, a transmission channel number. In the medical facility such as a hospital, one different channel is assigned to each transmitter 1, and it is assumed that a correspondence relationship between the channel and the transmitter is not changed. Therefore, in the medical facility such as a hospital, for example, the transmitter 1 can be specified by specifying the channel. Therefore, in a medical facility such as a hospital, for example, the subject can be specified by specifying the channel. In addition, for example, when a certain transmitter 1 is desired to be used in a certain hospital ward, it is necessary to register information (specifically, the second specifying information to be described later) indicating a channel used when the transmitter 1 transmits information in the reception device 2 in the hospital ward in advance. Therefore, a large number of channels (channels assigned to the transmitter 1) are registered in the reception device 2. A registration status of the channel in the reception device 2 is appropriately changed for each hospital ward according to entering and leaving a hospital of the subject. The first identification information is, for example, a hospital ID preset for the transmitter 1. Note that the hospital ID is an identifier indicating a medical facility, a medical department, or the like, and is an identifier for preventing erroneous display of physiological information transmitted from another hospital via the same communication path. The hospital ID is represented by a number such as “1”, for example.

In the present embodiment, the physiological information is classified into genuine physiological information that is normally transmitted and received and non-genuine physiological information that is not normally transmitted and received. The “genuine physiological information that is normally transmitted and received” here is physiological information transmitted from the transmitter 1 to the reception device 2 via the preset communication path and received by the reception device 2. On the other hand, the “non-genuine physiological information that is not normally transmitted and received” is, for example, physiological information corresponding to noise affected by so-called body motion or the like, or physiological information that is transmitted at a frequency used in communication performed between the transmitter 1 and the reception device 2 but should not be transmitted to the reception device 2 in the first place. Therefore, in the present embodiment, for example, even physiological information indicating a value outside an appropriate range is genuine physiological information if the physiological information is transmitted from the transmitter 1 to the reception device 2 via the preset communication path and is received by the reception device 2. That is, for example, physiological information representing arrhythmia or the like may be genuine physiological information.

When the transmitter 1 transmits the physiological information to the reception device 2 for a predetermined time or more, the transmitter 1 generates, for example, flag information as information indicating that the transmitter 1 has transmitted the physiological information to the reception device 2 for the predetermined time or more. The information indicating that the transmitter 1 has transmitted the physiological information to the reception device 2 for the predetermined time or more is not limited to the flag information. The transmitter 1 transmits the generated flag information to the reception device 2.

The reception device 2 is, for example, a central monitor. The reception device 2 can communicate with the transmitter 1. The reception device 2 can include an input and output interface 21, a storage device 22, and a controller 23. These components are communicably connected to each other via a bus 24.

The input and output interface 21 functions as an interface between the transmitter 1 and the reception device 2.

The storage device 22 is configured to store, for example, various data. The storage device 22 is a storage device such as a hard disk drive (HDD), a solid state drive (SSD), or a flash memory. In the storage device 22, for example, second specifying information for specifying a communication path that can be used by the reception device 2 and second identification information for specifying the transmitter 1 that can communicate with the reception device 2 are stored in advance. The second specifying information is, for example, information indicating a channel or a frequency that can be used when the reception device 2 receives the information from the transmitter 1 using radio waves. The second identification information is, for example, a hospital ID preset for the reception device 2. When the communication path specified by the first specifying information matches the communication path specified by the second specifying information, the reception device 2 can receive various types of information from the transmitter 1.

The controller 23 can includes a memory 231 and a processor 232, as a hardware configuration. For example, the memory 231 can include a read only memory (ROM) configured to store various computer programs, and a random access memory (RAM) having a plurality of work areas in which various computer programs to be executed by the processor 232 are stored. The processor 232 is, for example, a central processing unit (CPU), and is configured to load a computer program specified from various computer programs incorporated in the ROM on the RAM and execute various processes in cooperation with the RAM.

In the present embodiment, a computer readable medium may be used. The computer readable medium refers to a type of physical memory (RAM, ROM, or the like) that may store information and data readable by the processor. The computer readable medium may store instructions related to an execution process by one or more processors. The term “computer readable medium” can include a tangible item and excludes a carrier wave or a temporary signal (that is, refers to a non-transitory medium). Examples of the non-transitory computer readable medium can include a magnetic recording medium (for example, a flexible disk, a magnetic tape, or a hard disk drive), a magneto-optical recording medium (for example, a magneto-optical disk), a CD read only memory (CD-ROM), a CD-R, a CD-R/W, and a semiconductor memory (for example, a mask ROM, a programmable ROM (PROM), an erasable PROM (EPROM), or a flash ROM).

When the communication path specified by the first specifying information matches the communication path specified by the second specifying information, the controller 23 determines whether the first identification information is received from the transmitter 1. When the communication path specified by the first specifying information matches the communication path specified by the second specifying information, the controller 23 determines whether the transmitter 1 specified by the first identification information matches the transmitter 1 specified by the second identification information. When it is determined that the first identification information has not been received from the transmitter 1 or that the transmitter 1 specified by the first identification information does not match the transmitter 1 specified by the second identification information, the controller 23 determines that interference is occurring. Then, the controller 23 generates interference information indicating that interference is occurring based on the determination that interference is occurring. The controller 23 transmits the interference information to the storage device 22.

When the transmitter 1 specified by the first identification information matches the transmitter 1 specified by the second identification information, the controller 23 determines whether the physiological information has been transmitted from the transmitter 1 and received by the reception device 2 for the predetermined time or more. At this time, when it is determined that the physiological information has been transmitted from the transmitter 1 and received by the reception device 2 for the predetermined time or more, the controller 23 generates transmission/reception confirmation information indicating that the physiological information has been transmitted from the transmitter 1 and received by the reception device 2 for the predetermined time or more. The controller 23 performs the bed use process for starting monitoring of the physiological information on the subject based on the transmission/reception confirmation information. As described above, the transmission/reception confirmation information can be information serving as a trigger for the controller 23 to start the bed use process. That is, in the present embodiment, the controller 23 performs the bed use process with the generation of the transmission/reception confirmation information as a trigger. Therefore, in the present embodiment, it can be said that the controller 23 performs the bed use process when it is determined that the physiological information has been transmitted from the transmitter 1 and received by the reception device 2 for the predetermined time or more. The bed use process is to start monitoring of physiological information on the subject, and when the bed use process is performed, for example, biological waveform information on the subject, a measurement value (an example of the physiological information on the subject) in a medical device or the like, and the like are displayed on a display unit of the central monitor (an example of the reception device 2). In addition, when the bed use process is performed, a state of the central monitor becomes a state in which the medical worker or the like can input information such as the height and the weight of the subject on the bed use screen displayed on the display unit of the central monitor.

The controller 23 may be configured to cause the state of the reception device 2 to transition from a first state to a second state when the transmitter 1 specified by the first identification information matches the transmitter 1 specified by the second identification information. The first state is a state in which the flag information cannot be received from the transmitter 1, and the second state is a state in which flag information can be received from the transmitter 1. In this case, when the state of the reception device 2 is the second state, the controller 23 may determine whether the reception device 2 has received the flag information from the transmitter 1. In this case, the controller 23 performs the bed use process for starting monitoring of the physiological information on the subject based on the flag information received from the transmitter 1. That is, in this case, when it is determined that the reception device 2 has received the flag information from the transmitter 1 while the state of the reception device 2 is the second state, the controller 23 performs the bed use process.

### First Example of First Embodiment

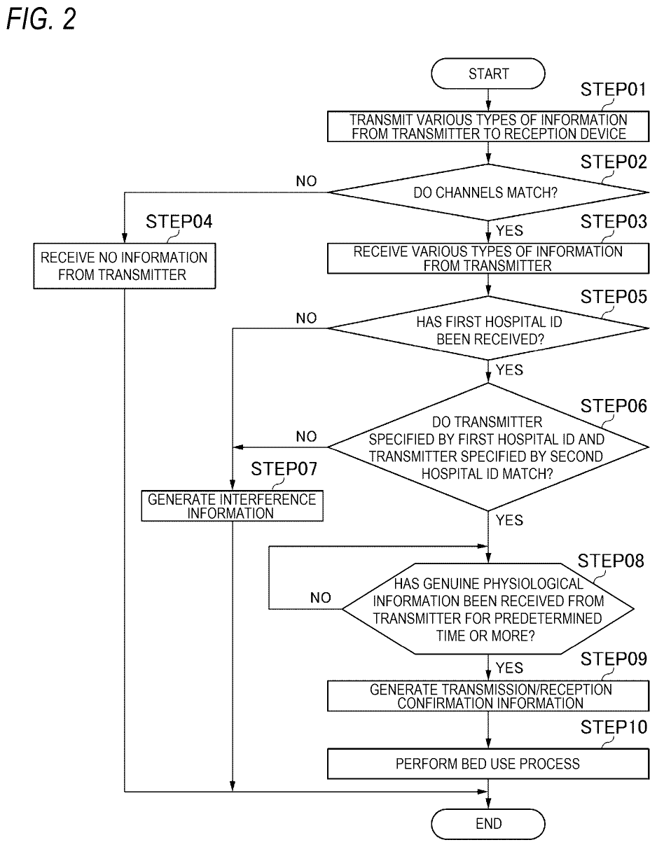

Next, an information processing method performed in a first example of the first embodiment will be described with reference to FIG. 2. FIG. 2 is a flowchart illustrating the information processing method performed in the first example of the first embodiment.

In the present example, when the transmitter 1 specified by the first identification information matches the transmitter 1 specified by the second identification information, the controller 23 determines whether the reception device 2 has received the genuine physiological information from the transmitter 1 for the predetermined time or more. Note that, in the present example, the first identification information and the second identification information are referred to as the “hospital IDs”, the hospital ID corresponding to the first identification information is referred to as a “first hospital ID”, and the hospital ID corresponding to the second identification information is referred to as a “second hospital ID”. In present example, the predetermined time is 10 seconds. However, the predetermined time is not limited to this example. When it is determined that the reception device 2 has received the genuine physiological information from the transmitter 1 for the predetermined time or more, the controller 23 generates the transmission/reception confirmation information. Therefore, the transmission/reception confirmation information generated by the controller 23 in the present example is information indicating that the genuine physiological information has been transmitted from the transmitter 1 and received by the reception device 2 for the predetermined time or more.

As illustrated in FIG. 2, the transmitter 1 transmits various types of information to the reception device 2 (STEP01). The various types of information transmitted by the transmitter 1 to the reception device 2 in STEP01 are, for example, the physiological information, the first specifying information, and the first hospital ID, or the physiological information and the first specifying information. The physiological information transmitted by the transmitter 1 to the reception device 2 in STEP01 includes the genuine physiological information.

When a channel (an example of the communication path) specified by the first specifying information matches a channel specified by the second specifying information (YES in STEP02), the reception device 2 receives various types of information transmitted from the transmitter 1 (STEP03). On the other hand, when the channel specified by the first specifying information does not match the channel specified by the second specifying information (NO in STEP02), the reception device 2 does not receive the various types of information transmitted from the transmitter 1 (STEP04). In this case, a subsequent information process is not performed.

After STEP03, the controller 23 of the reception device 2 determines whether the reception device 2 has received the first hospital ID from the transmitter 1 (STEP05). That is, the controller 23 determines whether the various types of information received from the transmitter 1 include the first hospital ID. When the reception device 2 has received the first hospital ID from the transmitter 1 (YES in STEP05), the controller 23 executes STEP06. On the other hand, when the reception device 2 has not received the first hospital ID from the transmitter 1 (NO in STEP05), the controller 23 determines that interference is occurring, and generates the interference information based on the determination (STEP07).

In STEP06, the controller 23 determines whether the transmitter 1 specified by the first hospital ID matches the transmitter 1 specified by the second hospital ID. When the transmitter 1 specified by the first hospital ID matches the transmitter 1 specified by the second hospital ID (YES in STEP06), the controller 23 executes STEP08. On the other hand, when the transmitter 1 specified by the first hospital ID does not match the transmitter 1 specified by the second hospital ID (NO in STEP06), the controller 23 generates the interference information (STEP07).

In STEP08, the controller 23 determines whether the reception device 2 has received the genuine physiological information from the transmitter 1 for the predetermined time (10 seconds in the present example) or more. When it is determined that the reception device 2 has received the genuine physiological information from the transmitter 1 for the predetermined time or more (YES in STEP08), the controller 23 generates the transmission/reception confirmation information (STEP09). Then, when the transmission/reception confirmation information is generated, the controller 23 performs the bed use process based on the generated transmission/reception confirmation information (STEP10). That is, the controller 23 performs the bed use process with the generation of the transmission/reception confirmation information as a trigger. On the other hand, when it is determined that the reception device 2 has not received the genuine physiological information from the transmitter 1 for the predetermined time or more (NO in STEP08), the controller 23 executes STEP08 again.

According to the reception device 2 having the above configuration, for example, even if the medical worker does not perform a predetermined input for performing the bed use process on the reception device 2, the reception device 2 performs the bed use process when it is determined that the physiological information has been transmitted from the transmitter 1 and received by the reception device 2 for the predetermined time or more when the transmitter 1 specified by the first hospital ID (the first identification information) matches the transmitter 1 specified by the second hospital ID (the second identification information). As described above, according to the reception device 2, it is possible to reduce the burden on the medical worker related to the bed use process. In addition, the information processing method, the computer program and the non-transitory computer readable medium according to the above configurations can also achieve the same or similar effects.

According to the reception device 2 having the above configuration, since the bed use process is performed when it is determined that the genuine physiological information has been transmitted from the transmitter 1 and received by the reception device 2 for the predetermined time or more, the bed use process can be performed more accurately.

According to the reception device 2 having the above configuration, since the bed use process has been performed when it is determined that the physiological information is received from the transmitter 1 for the predetermined time or more, for example, even if the configuration of the transmitter 1 is simple, the reception device 2 can automatically perform the bed use process.

### Second Example of First Embodiment

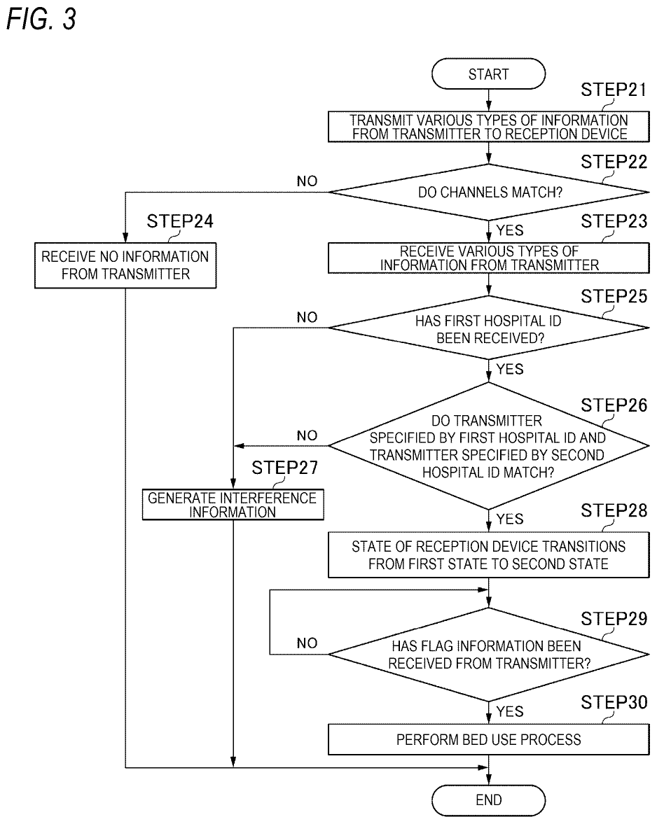

Next, an information processing method performed in a second example of the first embodiment will be described with reference to FIG. 3. FIG. 3 is a flowchart illustrating the information processing method executed in the second example of the first embodiment.

In the present example, when the transmitter 1 transmits the genuine physiological information to the reception device 2 for the predetermined time (10 seconds in the present example) or more, the transmitter 1 generates the flag information as information indicating that the transmitter 1 has transmitted the genuine physiological information to the reception device 2 for the predetermined time (10 seconds in the present example) or more. Further, in the present example, when the transmitter 1 specified by the first hospital ID (the first identification information) matches the transmitter 1 specified by the second hospital ID (the second identification information), the controller 23 causes the state of the reception device 2 to transition from the first state to the second state. Furthermore, in the present example, when the state of the reception device 2 is the second state, the controller 23 determines whether the reception device 2 has received the flag information from the transmitter 1. When it is determined that the reception device 2 has received the flag information from the transmitter 1, the controller 23 performs the bed use process based on the flag information received from the transmitter 1. That is, the present example is different from the first example of the first embodiment in that the controller 23 of the reception device 2 performs the bed use process based on the flag information instead of the transmission/reception confirmation information.

As illustrated in FIG. 3, STEP21 to STEP27 are the same as or similar to STEP01 to STEP07 in the first example of the first embodiment. In the present example, when the transmitter 1 specified by the first hospital ID matches the transmitter 1 specified by the second hospital ID (YES in STEP26), the state of the reception device 2 transitions from the first state to the second state (STEP28).

The controller 23 of the reception device 2 determines whether the reception device 2 has received the flag information from the transmitter 1 (STEP29). When the controller 23 determines that the reception device 2 has received the flag information from the transmitter 1 (YES in STEP29), the controller 23 performs the bed use process (STEP30). On the other hand, when the controller 23 determines that the reception device 2 has not received the flag information from the transmitter 1 (NO in STEP29), the controller 23 executes STEP29 again.

Also in this example, the reception device 2 has the same or similar effect as that of the first example of the first embodiment.

According to the reception device 2 having the above configuration, when the reception device 2 receives, from the transmitter 1, the information (the flag information) indicating that the transmitter 1 has transmitted the physiological information to the reception device 2 for the predetermined time (10 seconds in the present example) or more, the reception device 2 performs the bed use process. That is, the reception device 2 does not need to generate the transmission/reception confirmation information at the time of the bed use process, and can perform the bed use process only by determining whether the flag information has been received from the transmitter 1. Therefore, according to the reception device 2 having the above configuration, it is possible to reduce a load of information processing performed by the reception device 2.

### Third Example of First Embodiment

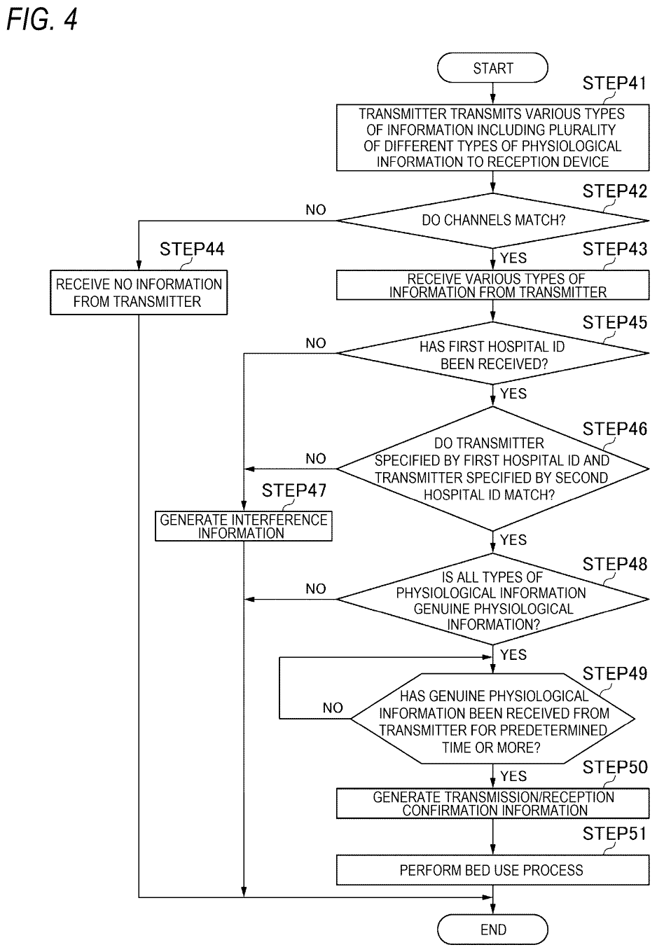

Next, an information processing method performed in a third example of the first embodiment will be described with reference to FIG. 4. FIG. 4 is a flowchart illustrating the information processing method performed in the third example of the first embodiment.

In the present example, the reception device 2 receives a plurality of different types of physiological information (the electrocardiogram waveform data and the percutaneous arterial oxygen saturation in the present example) from the transmitter 1. In addition, in the present example, when all types of physiological information received from the transmitter 1 are the genuine physiological information and the genuine physiological information has been received from the transmitter 1 for the predetermined time or more, the controller 23 of the reception device 2 generates the transmission/reception confirmation information and performs the bed use process based on the transmission/reception confirmation information. That is, the present example is different from the first example of the first embodiment in that the controller 23 performs the bed use process when all types of physiological information are the genuine physiological information. Therefore, in the present example, the controller 23 determines whether all types of physiological information received from the transmitter 1 are the genuine physiological information.

As illustrated in FIG. 4, the transmitter 1 transmits various types of information including a plurality of different types of physiological information to the reception device 2 (STEP41). In the present example, the transmitter 1 transmits two different types of physiological information (the electrocardiogram waveform data and the percutaneous arterial oxygen saturation) to the reception device 2.

STEP42 to STEP47 are the same as or similar to STEP01 to STEP07 in the first example of the first embodiment. In the present example, in STEP43, the reception device 2 receives various types of information including a plurality of different types of physiological information.

In STEP48, the controller 23 of the reception device 2 determines whether all types of physiological information received from the transmitter 1 are the genuine physiological information. When determining that all types of physiological information received from the transmitter 1 are the genuine physiological information (YES in STEP48), the controller 23 executes STEP49. On the other hand, when it is determined that at least one type of physiological information received from the transmitter 1 is not the genuine physiological information (NO in STEP48), the controller 23 ends the information processing.

STEP49 to STEP51 are the same as or similar to STEP08 to STEP10 in the first example of the first embodiment.

Also in this example, the reception device 2 has the same or similar effect as that of the first example of the first embodiment.

According to the reception device 2 having the above configuration, since the bed use process is performed when all types of physiological information received from the transmitter 1 are the genuine physiological information, the bed use process can be performed more accurately.

### Second Embodiment

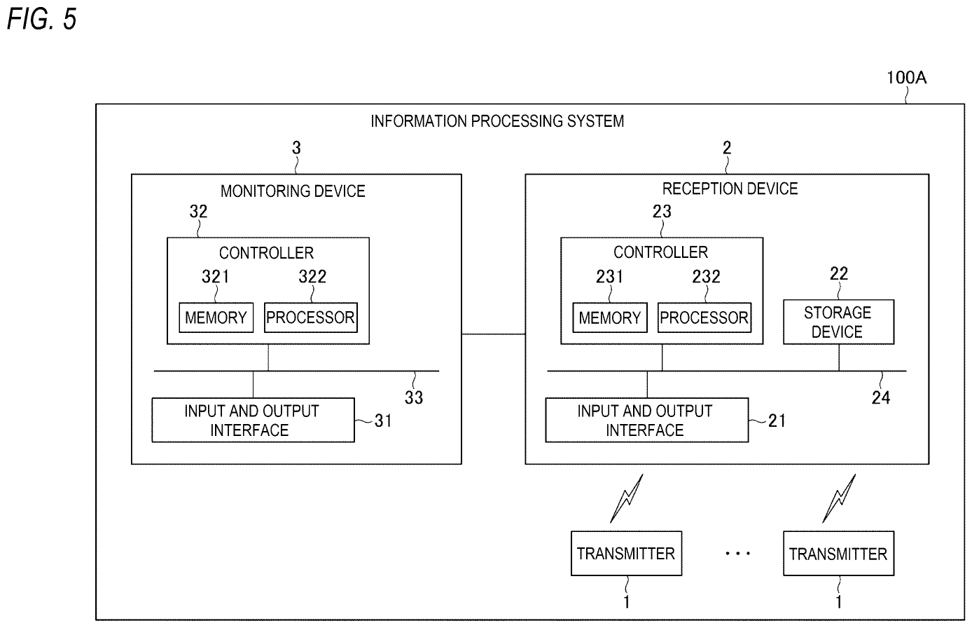

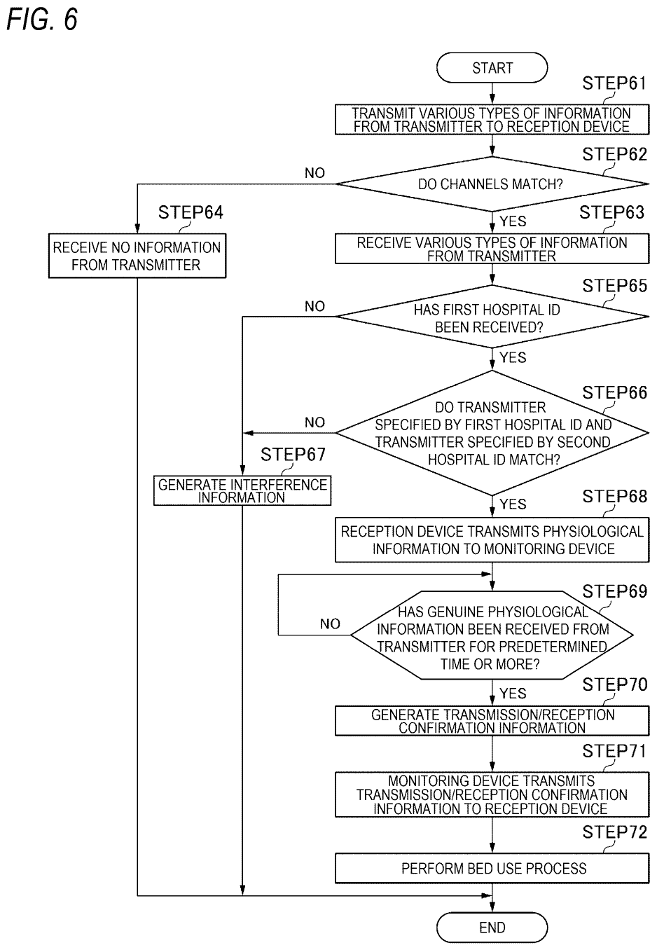

Next, an information processing system 100A according to a second embodiment will be described with reference to FIGS. 5 and 6. In the present embodiment, portions similar to those of the first embodiment will be described using the same reference numerals as those of the first embodiment, and description of overlapped portions will be appropriately omitted. FIG. 5 is a system configuration diagram of the information processing system 100A according to the second embodiment. FIG. 6 is a flowchart illustrating an information processing method performed in the second embodiment. As illustrated in FIG. 5, the information processing system 100A can include the transmitters 1, the reception device 2, and a monitoring device 3. In this respect, the information processing system 100A according to the present embodiment is different from the information processing system 100 according to the first embodiment in that the monitoring device 3 is provided. In the present embodiment, the input and output interface 21 of the reception device 2 functions not only as an interface between the transmitter 1 and the reception device 2 but also as an interface between the reception device 2 and the monitoring device 3.

As illustrated in FIG. 5, the monitoring device 3 is communicably connected to the reception device 2. In the present embodiment, the reception device 2 transmits, for example, the physiological information received from the transmitter 1 to the monitoring device 3. Therefore, in the present embodiment, the monitoring device 3 receives the physiological information from the reception device 2. In the present embodiment, the physiological information transmitted from the reception device 2 to the monitoring device 3 may include the genuine physiological information. The monitoring device 3 can includes an input and output interface 31 and a controller 32. The input and output interface 31 and the controller 32 are communicably connected via a bus 33.

The input and output interface 31 functions as an interface between the reception device 2 and the monitoring device 3.

The controller 32 can includes a memory 321 and a processor 322, as a hardware configuration. For example, the memory 321 can include a read only memory (ROM) configured to store various computer programs, and a random access memory (RAM) having a plurality of work areas in which various computer programs to be executed by the processor 322 are stored. The processor 322 is, for example, a central processing unit (CPU), and is configured to load a computer program specified from various computer programs incorporated in the ROM on the RAM and execute various processes in cooperation with the RAM.

The controller 32 determines whether the reception device 2 has received the genuine physiological information from the transmitter 1 for the predetermined time (for example, 10 seconds) or more. For example, when the monitoring device 3 receives the genuine physiological information from the reception device 2 for the predetermined time or more, the controller 32 determines that the reception device 2 has received the genuine physiological information from the transmitter 1 for the predetermined time or more.

When it is determined that the reception device 2 has received the genuine physiological information from the transmitter 1 for the predetermined time or more, the controller 32 generates the transmission/reception confirmation information. The controller 32 transmits the generated transmission/reception confirmation information to the reception device 2. Therefore, in the present embodiment, the reception device 2 receives the transmission/reception confirmation information from the monitoring device 3. In addition, the reception device 2 in the present embodiment performs the bed use process based on the transmission/reception confirmation information received from the monitoring device 3. That is, in the present embodiment, when it is determined that the transmission/reception confirmation information has been received from the monitoring device 3, the controller 23 performs the bed use process.

In the present embodiment, an information processing method illustrated in FIG. 6 is performed. STEP61 to STEP67 are the same as or similar to STEP01 to STEP07 in the first example of the first embodiment.

The reception device 2 transmits the physiological information received from the transmitter 1 to the monitoring device 3 (STEP68).

The controller 32 of the monitoring device 3 determines whether the reception device 2 has received the genuine physiological information from the transmitter 1 for the predetermined time or more (STEP69). When it is determined that the reception device 2 has received the genuine physiological information from the transmitter 1 for the predetermined time or more (YES in STEP69), the controller 32 generates the transmission/reception confirmation information (STEP70). On the other hand, when it is determined that the reception device 2 has not received the genuine physiological information from the transmitter 1 for the predetermined time or more (NO in STEP69), the controller 32 executes STEP69 again.

The monitoring device 3 transmits the generated transmission/reception confirmation information to the reception device 2 (STEP71).

STEP72 is the same as or similar to STEP10 in the first example of the first embodiment. In the present embodiment, the controller 23 performs the bed use process based on the transmission/reception confirmation information received from the monitoring device 3.

Also in the present embodiment, the reception device 2 has the same or similar effect as that of the first embodiment.

According to the reception device 2 having the above configuration, the reception device 2 executes the bed use process by receiving the transmission/reception confirmation information from the monitoring device 3. That is, according to the reception device 2 having the above configuration, the reception device 2 does not need to generate the transmission/reception confirmation information at the time of the bed use process, and performs the bed use process when the transmission/reception confirmation information is received from the monitoring device 3, so that it is possible to reduce a load of the information processing performed by the reception device 2.

The above embodiments are intended to facilitate understanding of the presently disclosed subject matter, and are not intended to limit the presently disclosed subject matter. The presently disclosed subject matter may be modified or improved without departing from the gist thereof.

In the first example of the first embodiment, the controller 23 of the reception device 2 generates the transmission/reception confirmation information when it is determined that the reception device 2 has received the genuine physiological information from the transmitter 1 for the predetermined time or more, but the presently disclosed subject matter is not limited to this example. For example, the controller 23 may generate the transmission/reception confirmation information when it is determined that the reception device 2 has received the physiological information from the transmitter 1 for the predetermined time or more. That is, in this case, the controller 23 generates the transmission/reception confirmation information even when it is determined that the reception device 2 has received the non-genuine physiological information from the transmitter 1 for the predetermined time or more. Same or similarly, in the second embodiment, the controller 32 of the monitoring device 3 may generate the transmission/reception confirmation information when it is determined that the reception device 2 has received the physiological information from the transmitter 1 for the predetermined time or more.

In the second example of the first embodiment, the transmitter 1 generates the flag information when the transmitter 1 transmits the genuine physiological information to the reception device 2 for the predetermined time or more, but the presently disclosed subject matter is not limited to this example. For example, the transmitter 1 may generate the flag information when the transmitter 1 transmits the physiological information to the reception device 2 for the predetermined time or more. That is, in this case, the transmitter 1 generates the flag information even when the transmitter 1 transmits the non-genuine physiological information to the reception device 2 for the predetermined time or more.

In the third example of the first embodiment, two different types of physiological information (the electrocardiogram waveform data and the percutaneous arterial oxygen saturation) are used, but three or more different types of physiological information may be used.

As described above, the present specification discloses items described in the following clauses.

### Clause 1

A reception device configured to communicate with at least one transmitter,
wherein the reception device is configured to receive, from the at least one transmitter, at least first specifying information for specifying a communication path used when the transmitter transmits information to the reception device using radio waves, first identification information for specifying the at least one transmitter, and physiological information on a subject associated with the at least one transmitter,
wherein the reception device is configured to store second specifying information for specifying a communication path that is used by the reception device, and second identification information for specifying the at least one transmitter that is communicated with the reception device,
wherein the reception device is configured to receive the physiological information from the at least one transmitter when the communication path specified by the first specifying information matches the communication path specified by the second specifying information, and
wherein, when the at least one transmitter specified by the first identification information matches the at least one transmitter specified by the second identification information, the reception device is configured to perform a bed use process for starting monitoring of the physiological information on the subject based on information that determines that the physiological information has been transmitted from the at least one transmitter and received by the reception device for a predetermined time or more.

### Clause 2

The reception device according to clause 1,
wherein the physiological information is classified into genuine physiological information that is normally transmitted and received and non-genuine physiological information that is not normally transmitted and received, and
wherein the bed use process is performed based on information that determines that the genuine physiological information has been transmitted from the at least one transmitter and received by the reception device for a predetermined time or more.

### Clause 3

The reception device according to clause 2, in which
a plurality of different types of the physiological information is received from the at least one transmitter, and
the bed use process is performed when all types of the physiological information are the genuine physiological information.

### Clause 4

The reception device according to any one of clauses 1 to 3, in which
the determination is a determination as to whether the reception device has received the physiological information from the at least one transmitter for the predetermined time or more.

### Clause 5

The reception device according to any one of clauses 1 to 4, in which
the determination is a determination as to whether the reception device has received, from the at least one transmitter, information indicating that the at least one transmitter has transmitted the physiological information to the reception device for the predetermined time or more.

### Clause 6

An information processing method performed by a reception device configured to communicate with at least one transmitter, the method comprising:
receiving physiological information from the at least one transmitter when a communication path specified by first specifying information matches a communication path specified by second specifying information; and
performing a bed use process for starting monitoring of the physiological information on a subject based on information that determines that the physiological information has been transmitted from the at least one transmitter and received by the reception device for a predetermined time or more, when the at least one transmitter specified by first identification information matches the at least one transmitter specified by second identification information,
wherein the reception device is configured to receive, from the at least one transmitter, at least the first specifying information for specifying the communication path used when the at least one transmitter transmits information to the reception device using radio waves, the first identification information for specifying the at least one transmitter, and the physiological information on the subject associated with the at least one transmitter, and
wherein the reception device is configured to store the second specifying information for specifying the communication path that is used by the reception device, and the second identification information for specifying the at least one transmitter that is communicated with the reception device.

### Clause 7

A computer program including at least one instruction to be executed by one or more processors of a reception device configured to communicate with at least one transmitter, when at least one instruction is executed by the one or more processors, the computer program causes the reception device to:
receive physiological information from the at least one transmitter when a communication path specified by first specifying information matches a communication path specified by second specifying information; and
perform a bed use process for starting monitoring of the physiological information on a subject based on information that determines that the physiological information has been transmitted from the at least one transmitter and received by the reception device for a predetermined time or more, when the at least one transmitter specified by first identification information matches the at least one transmitter specified by second identification information,
wherein the reception device is configured to receive, from the at least one transmitter, at least the first specifying information for specifying the communication path used when the at least one transmitter transmits information to the reception device using radio waves, the first identification information for specifying the at least one transmitter, and the physiological information on the subject associated with the at least one transmitter, and
wherein the reception device is configured to store the second specifying information for specifying the communication path that is used by the reception device, and the second identification information for specifying the at least one transmitter that is communicated with the reception device.

### Clause 8

A Non-transitory Computer Readable Medium Storing the Computer Program According to clause 7.

1. A reception device configured to communicate with at least one transmitter,
wherein the reception device is configured to receive, from the at least one transmitter, at least first specifying information for specifying a communication path used when the transmitter transmits information to the reception device using radio waves, first identification information for specifying the at least one transmitter, and physiological information on a subject associated with the at least one transmitter,
wherein the reception device is configured to store second specifying information for specifying a communication path that is used by the reception device, and second identification information for specifying the at least one transmitter that is communicated with the reception device,
wherein the reception device is configured to receive the physiological information from the at least one transmitter when the communication path specified by the first specifying information matches the communication path specified by the second specifying information, and
wherein, when the at least one transmitter specified by the first identification information matches the at least one transmitter specified by the second identification information, the reception device is configured to perform a bed use process for starting monitoring of the physiological information on the subject based on information that determines that the physiological information has been transmitted from the at least one transmitter and received by the reception device for a predetermined time or more.

2. The reception device according to claim 1,
wherein the physiological information is classified into genuine physiological information that is normally transmitted and received and non-genuine physiological information that is not normally transmitted and received, and
wherein the bed use process is performed based on information that determines that the genuine physiological information has been transmitted from the at least one transmitter and received by the reception device for a predetermined time or more.

3. The reception device according to claim 2, wherein
a plurality of different types of the physiological information is received from the at least one transmitter, and
the bed use process is performed when all types of the physiological information are the genuine physiological information.

4. The reception device according to claim 1, wherein
the determination is a determination as to whether the reception device has received the physiological information from the at least one transmitter for the predetermined time or more.

5. The reception device according to claim 1, wherein
the determination is a determination as to whether the reception device has received, from the at least one transmitter, information indicating that the at least one transmitter has transmitted the physiological information to the reception device for the predetermined time or more.

6. An information processing method performed by a reception device configured to communicate with at least one transmitter, the method comprising:
receiving physiological information from the at least one transmitter when a communication path specified by first specifying information matches a communication path specified by second specifying information; and
performing a bed use process for starting monitoring of the physiological information on a subject based on information that determines that the physiological information has been transmitted from the at least one transmitter and received by the reception device for a predetermined time or more, when the at least one transmitter specified by first identification information matches the at least one transmitter specified by second identification information,
wherein the reception device is configured to receive, from the at least one transmitter, at least the first specifying information for specifying the communication path used when the at least one transmitter transmits information to the reception device using radio waves, the first identification information for specifying the at least one transmitter, and the physiological information on the subject associated with the at least one transmitter, and
wherein the reception device is configured to store the second specifying information for specifying the communication path that is used by the reception device, and the second identification information for specifying the at least one transmitter that is communicated with the reception device.

7. A non-transitory computer readable medium storing a computer program including at least one instruction to be executed by one or more processors of a reception device configured to communicate with at least one transmitter, when at least one instruction is executed by the one or more processors, the computer program causes the reception device to:
receive physiological information from the at least one transmitter when a communication path specified by first specifying information matches a communication path specified by second specifying information; and
perform a bed use process for starting monitoring of the physiological information on a subject based on information that determines that the physiological information has been transmitted from the at least one transmitter and received by the reception device for a predetermined time or more, when the at least one transmitter specified by first identification information matches the at least one transmitter specified by second identification information,
wherein the reception device is configured to receive, from the at least one transmitter, at least the first specifying information for specifying the communication path used when the at least one transmitter transmits information to the reception device using radio waves, the first identification information for specifying the at least one transmitter, and the physiological information on the subject associated with the at least one transmitter, and
wherein the reception device is configured to store the second specifying information for specifying the communication path that is used by the reception device, and the second identification information for specifying the at least one transmitter that is communicated with the reception device.
